(19) United States
(12) Patent Application Publication
(10) Pub. No.: US 2006/0078591 A1
(43) Pub. Date: Apr. 13, 2006

(54) Systems and methods for augmenting tissue volume

(76) Inventor: Daniel Alexander Del Vecchio, Wrentham, MA (US)

Correspondence Address: David M. Driscoll, 1201 Canton Avenue, Milton, MA, 02186, US

(21) Appl. No.: 11/244,321

(22) Filed: Oct. 5, 2005

Related U.S. Application Data
(60) Provisional application No. 60/617,448, filed on Oct. 8, 2004.

(51) Int. Cl. A61K 35/54; A61F 2/00

(52) U.S. Cl. 424/426; 424/582

(57) ABSTRACT

A method of treating volume deficiency in cosmetic surgery and in urological stress incontinence is based upon the transplantation of one or more biologic collagen based tissues into the subcutaneous, deep dermal, or peri urethral space. In the preferred embodiment, mechanically morsellized dehydrated embryonic bovine matrix tissue is used, which is derived from commercially available sheets of lyophilized fetal bovine collagen. The specific morsellization, cubing and hydration techniques described herein allow the percutaneous insertion of the transplanted collagen tissue through a syringe using a needle. Morsellization and percutaneous injection also decreases the insertion requirements of the material transferred, thus allowing the surgeon to insert more material into the desired space with less morbidity. Once in the body, the collagen matrix acts as a scaffold to allow the ingrowth of host human fibroblasts. One or more therapeutic substances may be added, including growth factors, differentiation factors, hydrogels, polymers, antibiotics, anti-inflammatory medications, or immunosuppressive medications. These additional substances may or may not be dehydrated.

### RELATED APPLICATIONS

The present application claims priority to U.S. Provisional Application Ser. No. 60/617,448 filed on Oct. 8, 2004.

### BACKGROUND

1. Technical Field

The present invention relates generally to the treatment of soft tissue volume deficiency and loss, facial wrinkles, facial lines, insufficient penile girth and the treatment of urinary stress incontinence by peri urethral bulking and more particularly, to treatments for volume restoration using embryonic bovine matrix (EBM), autograft dermis or allograft dermis.

2. Background of the Invention

Contour defects and other volume abnormalities in soft tissues represent a common problem for plastic, reconstructive, dermatological and other surgeons. Techniques existing in the art offer solutions with recognized drawbacks, including transience of volume filling, local reactions and undesirable donor sites. Therefore, there remains a need in the art for more satisfactory systems and methods for tissue volume filling and augmentation.

There is currently no perfect volume filler material for cosmetic and reconstructive soft tissue augmentation. Commercially available bovine collagen is derived from enzymatic degradation from bovine hides and lacks structural cross linking. Such collagen lasts as an implant for 4-6 weeks. There is also a concern over the possibility of transmission of Mad Cow Disease (“MCD”) using bovine collagen preparations. Human collagen, derived from human fibroblasts in culture, is likewise enzymatically processed to rid cellular material and lasts a similarly short period of time in the body. In the case of human collagen, there is a small theoretical potential for viral transmission.

Restylane, a hyaluronic acid preparation, is FDA approved in the United States to augment the deep dermis and lasts 4-6 months. Drawbacks relating to Restylane include its cost per cc and the need for multiple (4-8) ccs of volume filler in many clinical cases.

Autologous fat is also used to inject into the subcutaneous space and lasts 4-6 months. This method of subcutaneous filler augmentation requires a donor site harvesting procedure and involves over-correction at the insertion site, as there is considerable resorption of fat volume early on due to the destruction of damaged fat cells during the process. This leads to considerable short-term morbidity.

Hydroxyappetite crystals are FDA approved in solid block form for bone interposition grafting. This product is used off-label, and is injected subcutaneously to treat deep facial lines. Persistence of volume has been demonstrated for 12-14 months. Such material is not soft and is easily palpable in vivo and can cause granulomas if injected too superficially. It is not FDA approved for this use.

More permanent fillers such as Gore Tex are inserted as strips subcutaneously and can serve as a nidus for bacteria. Because they are solid sheets they do not allow for dispersion in the subcutaneous space and can cause visible sharp edges under the skin.

Medical grade silicone gel has historically been used as a filler but its use is currently condemned due to long term problems with granuloma formation and an unclear causal association with connective tissue diseases. Fragmented solid silicone microspheres are currently undergoing FDA evaluation for use in bulking the peri-urethral soft tissues in the treatment of urinary stress incontinence.

Embryonic Bovine Collagen Matrix (EBM) has existing uses in the art for soft tissue structural reinforcement, providing structural stability to tissues subjected to abnormal or physiological stress, but has not been used for volume restoration. Fetal Collagen, or Embryonic Bovine Matrix (“EBM”) has been demonstrated to be effective as a soft tissue structural support in orthopedic ligament reconstruction, hernia repair and in wound closure. EBM is fetal Type I collagen and as such lacks immunogenicity. Because it is not enzymatically digested in its preparation to rid cellular material (U.S. Pat. No. 6,696,074 B2, Feb. 24, 2004), the sheets of EBM consist of collagen with retained cross linking and matrix architecture, and do not appear to have issues related to the risk of MCD. This is in sharp contrast to commercially injectable human or bovine collagen which is manufactured by enzymatic digestion of dermis, with glutyraldehyde processing, impeding recipient site cellular in growth.

### SUMMARY OF THE INVENTION

In accordance with the present invention there is provided a method for augmenting volume of a soft tissue. This method involves the delivery of Embryonic Bovine Collagen Matrix (EBM) to the soft tissue, or alternatively, autograft dermis or allograft dermis. The method involves the micronizing of the material, preferably morsellizing, fragmenting or cubing the material. The delivery step may be performed by a tamponade technique or a syringe injection technique. In one disclosed embodiment the injection material is morsellized, while in another embodiment it is fragmented, such as by cubing, before delivery it to the soft tissue. The soft tissue is selected from the group consisting of subdermal space, subcutaneous space, submucosal space, periurethral space and hypopharyngeal space.

In accordance with one aspect of the present invention there is provided a method of treating subcutaneous volume deficiency in, for example, the face, lips, cheeks, penis, and peri-urethral areas comprising the steps of processing sheets of dehydrated embryonic bovine matrix; morsellizing the material employing manual or machine operated mechanical methods or apparatus, rehydrating the morsellized tissues; and transplanting the re-hydrated tissue into the area being treated, such as via syringe injection.

In accordance with another aspect of the present invention there is provided a method of treating subcutaneous volume deficiency in, for example, the face, lips, cheeks, penis, and peri-urethral areas comprising the steps of: processing sheets of dehydrated embryonic bovine matrix, autograft dermis or allograft dermis; cubing the material employing manual or machine operated mechanical methods or apparatus, re-hydrating the cubed tissue; and transplanting the re-hydrated tissue into the area being treated.

In accordance with other aspects of the present invention one or more therapeutic substances may be added to the dehydrated tissue prior to the implantation thereof. The therapeutic substances may include one or more of the following: growth factors, differentiation factors, hydrogels, polymers, antibiotics, anti-inflammatory medications, or immunosuppressive medications. The morsellized embryonic bovine matrix may be injected into the area being treated through a needle and syringe or small cannula. The morsellized embryonic bovine matrix may be percutaneously or transmucosally injected into the area being treated. The cubed embryonic bovine matrix, autograft dermis or allograft dermis may be transplanted into the area being treated via a tamponade introducer. The material may be percutaneously or transmucosally transplanted into the area being treated.

In accordance with another aspect of the present invention there is provided a device for morsellizing a soft tissue filler material, comprising two cutting plates attachable at an angle to each other and a cutter operatively connected to the cutting plates, wherein each of the two cutting plates has a series of grooves separated by a predetermined distance, each groove directing the cutter to cut the soft tissue material into fragments of a preselected size.

### BRIEF DESCRIPTION OF THE DRAWINGS

Numerous other objects, features and advantages of the present invention will now become apparent upon a reading of the following detailed description as taken in conjunction with the accompanying drawings, in which:

FIG. 1 is a perspective view illustrating the use of a rasp device that is used to morsellize tissue in accordance with the present invention;

FIG. 2 illustrates the use of a syringe for implantation;

FIG. 3 illustrates the device of the present invention for fragmenting the processed material,

FIG. 4 illustrates the tamponade device of the present invention; and

FIG. 5 are a series of photographs showing comparisons.

### DETAILED DESCRIPTION

The following description relates to systems and methods for using EBM, autograft dermis or allograft dermis as a filler material for augmenting tissue volume. As used herein, the term “augmenting” includes both restoring abnormal contours to a more normal state for cosmetic or reconstructive reasons, and adding to the volume of an existing soft tissue space for cosmetic or reconstructive reasons. As described herein, volume enhancement includes cosmetic subcutaneous and intramuscular volume enhancement of the face, lips, and cheeks, volume enhancement for the treatment of age related and pathologic soft tissue atrophy, cosmetic volume enhancement of the breast and penis, and bulking of the periurethral soft tissues for the treatment of urinary stress incontinence and for urinary incontinence post prostatectomy. Further disclosed herein are systems and methods for preparing EBM into forms suitable for augmenting tissue volume.

Morsellized, cubed or micronized EBM retains cross linked collagen and retains its matrix architecture, allowing the potential for recipient fibroblasts to undergo cellular in-growth and remodeling, native collagen formation, and a longer lasting or potentially permanent volume fill.

In one embodiment there is a method of mechanically micronizing, morsellizing, fragmenting or cubing embryonic bovine collagen matrix (“EBM”), using hand held or mechanical rotary milling and rasping techniques. These techniques can be implemented at the point of care setting in the operating room or procedure room under aseptic sterile conditions to process injectable EBM. Alternatively the EBM morsellization process can be mass-processed on an industrial level and can be commercially packaged in a sterile pre-filled syringe form. The method of preparing and implanting the processed EBM is used in conjunction with, for example, syringe injection or the use of an implantation tamponade devices, both of which are described hereinafter.

Throughout this description reference is made to the implantation of EbM, however, it is understood that the concepts of the present invention also apply to the processing of autograft dermis or allograft dermis.

This invention, in one use thereof, is directed to a method of treating a volume-deficient area in the subcutaneous or deep dermal region of the face, cheek, lips, penis or per-urethral tissues through the transplantation of morsellized, cubed or micronized embryonic bovine matrix into the subcutaneous, deep dermal or peri-urethral tissue. In the preferred embodiment, morsellized, cubed or micronized embryonic bovine matrix is used, which may be combined with extracellular matrix materials. In other embodiments autograft dermis or allograft dermis are implanted.

The following are steps taken in performing one aspect of the method of the present invention, particularly for morsellizing EBM. Later in this description are set forth steps for cubing the EBM.

Morsellization:

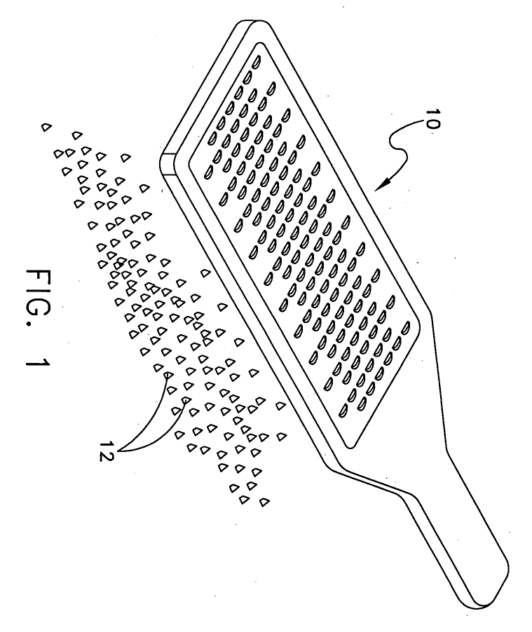

According to the method, embryonic bovine matrix is obtained in its sterile, commercially available sheet form from its manufacturer. Such sheets range in size depending on the clinical application, be it hernia repair, rotator cuff repair, or used as a sling for bladder suspension. The tissue is tightly rolled into a monobloc cylinder of 3-4 cm in length and 1-2 cm in diameter and is secured in this shape using, for example, sterile surgical clamps. The tight block of tissue is then introduced to a sterile hand-operated or machine operated rasping device, with blade openings on the order of 0.5 to 2 mm in size. Introducing the rolled edge of the monobloc to the blades of the rasp generates fragmentation and randomly variable morsellized microparticles of embryonic bovine matrix, ranging in size from 50-500 microns in diameter. FIG. 1 shows a rasp device 10 that can be used in performing the morsellizing. Because the embryonic bovine matrix is supplied lyophilized, such fragmentation is possible. The morsellized embryonic bovine matrix may then be collected and inserted into the barrel of a syringe using a sterile aseptic technique. FIG. 1 also illustrates, at 12, the processed particles from the rasping device 10.

Re-Hydration:

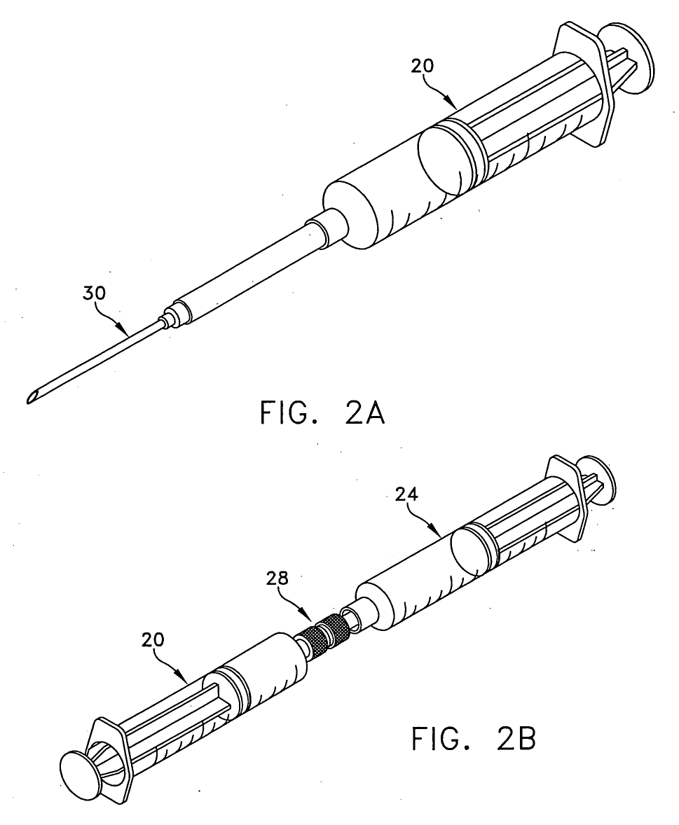

Once a sufficient volume of morsellized micronized embryonic bovine matrix is obtained in the barrel of a syringe, a female-female transfer hub is used to connect this syringe to a second syringe. The contents of this second syringe contain a volume of sterile normal saline, substantially equal to that of the volume of embryonic bovine matrix. Depression of the plungers in an alternate fashion allows for a mixture of low molecular weight salt and water molecules to inter-disperse between the spaces in the collagen matrix, yielding a volume of re-hydrated embryonic bovine matrix similar in volume to its dehydrated state. Refer to FIG. 2 for an illustration of a syringe apparatus that can be used in performing this re-hydration step. This includes a first syringe 20, a second syringe 24 and the female-to-female hub 28. Repeated transfer between the syringes is used to mix the final content. FIG. 2A also shows a needle 30 as attached to the output end of the syringe.

Injection:

Re-hydration allows the smooth gliding of the material through a 21 gauge or larger needle and the percutaneous insertion of the transplanted embryonic bovine matrix through a small percutaneous needle site. Re-hydration also allows for customization of flow by varying the amount of sterile saline to be used in the mixture. The use of less than equal parts of sterile saline/embryonic bovine matrix results in a thicker, more viscous injectable material that may be beneficial in deep tissues, whereas greater than equal parts of sterile saline/embryonic bovine matrix saline results in a more diluted mixture that flows through a smaller gauge needle, and can be used in a more superficial location in the subcutaneous space or deep dermis. Variations in the sterile saline/embryonic bovine matrix mixture allows alteration of the volume of the material transferred, thus allowing the surgeon to insert more or less embryonic bovine matrix into the subcutaneous, deep dermal or peri urethral space. Once in the body, the collagen matrix acts as a scaffold to allow the in-growth of host human fibroblasts.

An alternative embodiment includes the production and use of small, preferably 1-2 mm³ blocks, fragments or cubes, of embryonic bovine matrix (EBM). The cubing technique described herein may also be used in processing autograft dermis or allograft dermis.

Cubing:

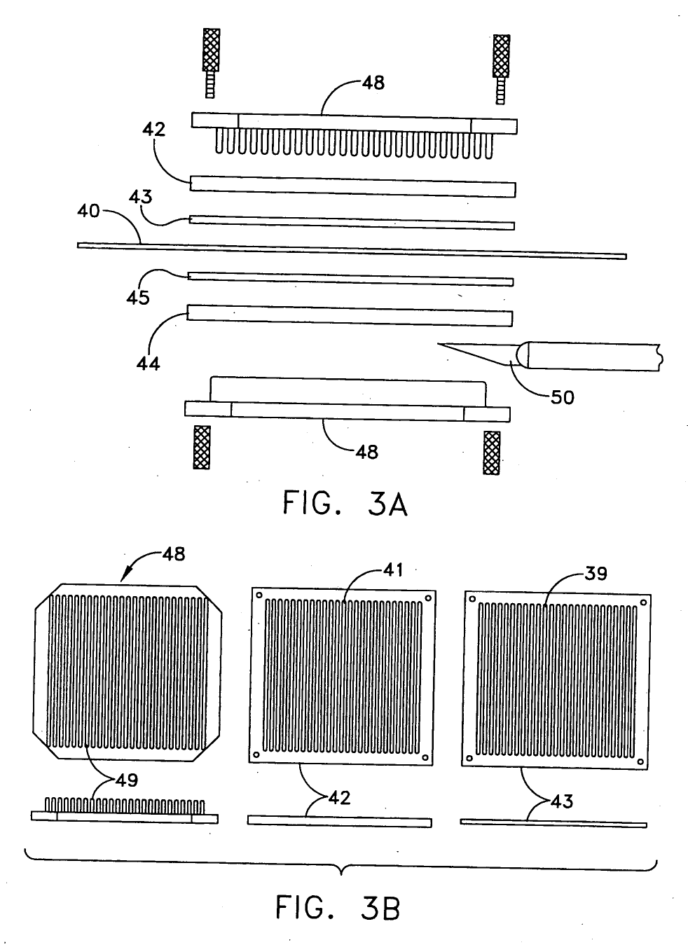

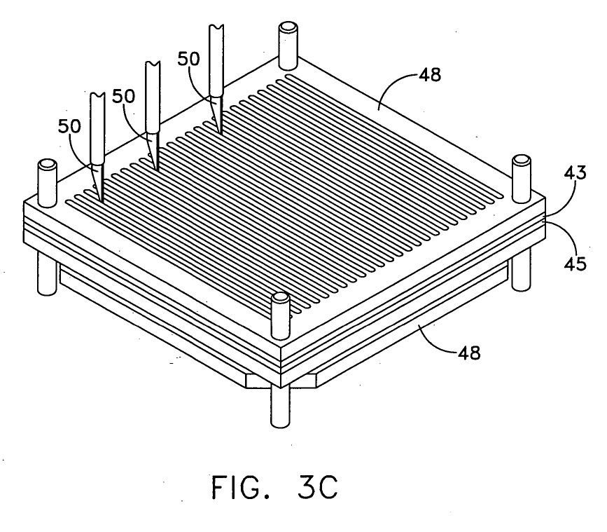

Embryonic bovine matrix is obtained in its sterile, commercially available sheet form from its manufacturer. Such sheets range in size depending on the clinical application, be it hernia repair, rotator cuff repair, or used as a sling for bladder suspension. The sheet tissue is introduced into a meshing device that consists of stainless steel plates with grids placed at right angles to one another. By tightly compressing the sheet of embryonic bovine matrix and cutting the embryonic bovine matrix on either side of the plates with a surgical blade, uniform blocks (particles) of embryonic bovine matrix are processed, varying in size with the thickness of the supplied embryonic bovine matrix and ranging from 1-2 mm³ in size. Refer to FIG. 3 for an illustration of a meshingor cubing device usable in performing this step and the resulting particles, respectively. A more detailed description of this unique meshing device is set forth hereinafter.

Re-Hydration:

Once a sufficient volume of cubed embryonic bovine matrix, autograft dermis or allograft dermis is obtained in the barrel of a syringe, a female-female transfer hub is used to connect this syringe to a second syringe. Again refer to FIG. 2. The contents of this second syringe contain a volume of sterile normal saline, approximately equal to that of the volume of embryonic bovine matrix. Depression of the plungers in an alternate fashion allows for a mixture of low molecular weight salt and water molecules to inter-disperse between the spaces in the collagen matrix, yielding a volume of re-hydrated embryonic bovine matrix similar in volume to its dehydrated state. The volume of saline can be adjusted greater or less than the volume of cubed embryonic bovine matrix in order to yield a less viscous or more viscous mixture, respectively. Variations in the sterile saline/cubed embryonic bovine matrix mixture allows alteration of the volume of the material transferred, thus allowing the surgeon to insert more or less embryonic bovine matrix into the subcutaneous, deep dermal or peri urethral space.

Implantation:

Re-hydration allows the smooth introduction of the cubed material into the barrel of a 16 gauge or larger tamponade needle and the subsequent percutaneous insertion of the transplanted embryonic bovine matrix through a small percutaneous opening, via a tamponade technique to be described in detail below. The surface to volume ratio of these cubes of embryonic bovine matrix is smaller compared to the surface to volume characteristics of the morsellized embryonic bovine matrix. This material is intended for use in the subcutaneous, intramuscular, or peri-urethral space where a large volume fill is required. It can also be used in the face in conjunction with smaller injections of morsellized embryonic bovine matrix, injected with a needle as previously described and used more superficially. Once in the body, the cubed collagen matrix acts as a scaffold to allow the in growth of host human fibroblasts.

Tamponade Insertion:

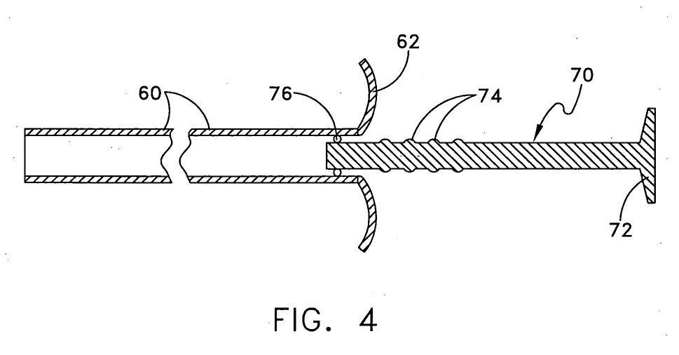

These cubes of embryonic bovine matrix, autograft dermis or allograft dermis do not inject well through a needle due to their size and flow characteristics. The use of a tamponade device is preferred. Refer to FIG. 4 for an illustration of a tamponade device used in the present invention. The tamponade device is a two component device consisting of a large gauge (12 to 16) blunt tipped tamponade needle, 45-60 mm in length, fitted with finger handles at the hub end. Along the inside lumen of the hub is a compressible spring loaded ball bearing that acts as a ratchet. The needle is attached by its hub end to a syringe containing hydrated, cubed embryonic bovine matrix. Using sufficient pressure in the syringe, a packed column of cubed hydrated embryonic bovine matrix is introduced into the needle. This fully-loaded needle is then removed and is ready to be used for tamponade insertion. Multiple needles of this type can be pre-loaded to facilitate the insertion process.

The second component of the tamponade insertion device is a tamponade plunger, a solid stainless steel rod that inserts flush into the lumen of the needle and is just as long. The tamponade plunger is fitted along its length with concentric notches at regular intervals of 2 mm. These notches engage the ball ratchet of the needle and generate a click and resistance to digital pressure during tamponade insertion of material. This resistance facilitated more precise amount of material fill with greater control. A more detailed description of this unique device is set forth hereinafter.

One or more therapeutic substances may be added during the insertion sequence, including growth factors, differentiation factors, hydrogels, polymers, antibiotics, or anti-inflammatory medications.

As discussed above, this invention resides in a method of treating a volume deficiency in the facial, penile or periurethral soft tissues through the processing and transplantation of one or more rehydrated biologic tissues into the subcutaneous, deep dermal, or periurethral space. In the preferred embodiment, rehydrated morsellized embryonic bovine matrix is used, which may be combined with rehydrated cubed embryonic bovine matrix. Due to mechanical as opposed to chemical processing, the relatively unchanged microscopic architecture of the processed embryonic bovine collagen matrix acts as a scaffold for recipient fibroblast in-growth. This invention provides an acellular collagen matrix material that is processed by mechanical morsellization or cubing and used in a morsellized or cubed fashion harvested from another human or animal.

The guidelines for the preparation of FDA-approved clinically available sheets of embryonic bovine matrix from fetal living tissues are well known to those skilled in the art. The text “Processing Fetal or Neo-Natal Tissue to Produce a Scaffold For Tissue Engineering” by Dai, et al., Feb. 24, 2004, describes such methods. Briefly, the tissue is to be devoid of all cellular material and be terminally sterilized prior to use. Examples of present preparation methods include: mechanical, treatment with sodium hydroxide wash, lyophilization, and gas sterilization using ethylene oxide. Details are provided in U.S. Pat. No. 6,696,074 B2, the entire contents of which are hereby incorporated by reference herein.

The aforementioned processed embryonic bovine collagen matrix is rehydrated as previously disclosed. To minimize bruising and bleeding in the subcutaneous tissues that could act as impediments to tissue in-growth into the scaffold, the embryonic bovine matrix is preferably inserted through a small hole in the skin using a blunt-tipped needle or cannula. Upon withdrawal of the needle, the material is injected, laying down, in some instances, parallel tubes of material into position, thereby increasing volume where desired.

The subcutaneous space in the facial region varies in fat content and age related and disease related volume loss also varies from individual to individual. Thus, in one embodiment, embryonic bovine collagen matrix is inserted into the nasolabial folds and lip and cheek regions, in varying amounts. The injection needle or tamponade device could be directed into the subcutaneous space percutaneously or transmucosally via stab incisions or needle insertion into the perioral or labial mucosa.

The rehydrated embryonic bovine collagen matrix may be morsellized to allow insertion into the subcutaneous or periurethral area through a small tamponade cannula or needle. With respect to the injectable processed embryonic bovine collagen matrix, the increased surface area after morsellization may also aid in the avoidance of lumpiness due to a smoother diversification of volume injected.

Once in the body, the embryonic bovine collagen matrix further hydrates by imbibing fluid from the surrounding area. In the case of the subcutaneous areas in the face, the subsequent hydration helps to restore subcutaneous volume and further enhance the treatment of the volume deficient area. Additional therapeutic substances may be added, including tissue growth or differentiation factors (recombinant generated morphogenetic proteins, PDGF, TGF-.beta., EGF/TGF-.alpha., IGF-I, PFGF), hydrogels, absorbable or nonresorbable synthetic or natural polymers (collagen, fibrin, polyglycolic acid, polylactic acid, polytetrafluoroethylene, etc.), antibiotics, anti-inflammatory medication, immunosuppressive medications, etc. could be beneficial. These additional substances may or may not contribute to rehydration, depending upon efficacy, initial versus final volume, and so forth.

Embryonic Bovine Collagen Matrix (EBM) may be used as a tissue volume filler in cosmetic and reconstructive surgery, in dermatology, in urology, in otolaryngology, and in similar medical specialties. According to the methods disclosed herein, this material may be used as a volume filler by surgical placement or subcutaneous tunneling. For example, EBM as a subdermal filler may be used in the form of a sheet or strip laid down subjacent to the nasolabial folds for treatment of depressions of the nasolabial folds, in the form of strips tunneled beneath the lip vermillion via stab incisions in the oral commissures, and in the form of sheets or strips placed subjacent to the corrugator muscles to replace volume in those procedures that weaken these muscles, for the purpose of reducing forehead wrinkles.

In other embodiments, the systems and methods disclosed herein may be used to place EBM, autograft dermis or allograft dermis subdermally via stab incisions in the skin to provide volume enhancement of the face in developmental maladies of facial fat and muscle volume atrophy. Conditions suitable for this treatment include, but are not limited to, Romberg's Hemifacial Atrophy, and facial lipodystrophy associated with HIV treatment, namely protease inhibitor therapy. In certain embodiments, the systems and methods disclosed herein may be used to place EBM subdermally in strips and pieces in the nasal region to provide volume enhancement of the nose in situations where nasal grafting is desired to provide volume enhancement. In certain embodiments, the systems and methods disclosed herein may be used to place EBM subdermally in strips and pieces in the cheek and peri-orbital region to provide volume enhancement of the cheek and peri-orbital soft tissues in situations where cheek and lower eyelid volume enhancement is desired to provide aesthetic enhancement. In certain embodiments, the systems and methods disclosed herein may be used to place EBM subdermally in the neck and midface region to provide mechanical suspension of the neck, cheek and peri-orbital soft tissues in situations where neck, cheek and peri-orbital soft tissues suspension and elevation is desired to provide aesthetic enhancement.

Embryonic Bovine Collagen Matrix (EBM) may further be conveniently prepared as a particulate filler material to be used for tissue volume augmentation in cosmetic and reconstructive surgery, in dermatology, in urology, in otolaryngology, and in similar medical specialties. For example, EBM may be morsellized into particles, cubes or other regular or irregular pieces, to be injected subdermally or intramuscularly via a needle using pressure from a syringe (Injectable Bovine Matrix, “IBM”). Injectable morsellized, cubed or particulate EBM can be placed subdermally or intramuscularly via needle insertion through the skin as sub dermal volume fillers in cosmetic and reconstructive surgery. Used in this manner, EBM provides volume replacement for contour defects and not structural reinforcement for load-bearing soft tissues. Such morsellized sub dermal fillers can take the form of cubes, spheres, or randomly cut shapes of EBM, ranging in volume between 0.001 mm³ and 27 mm³. In one embodiment, the morsellized EBM is placed via standard syringe injection after hydration with sterile saline subjacent to the nasolabial folds for treatment of depressions of the nasolabial folds, injected beneath the lip vermillion via stab incisions in the commissures, as currently performed using auto fat grafting in lip augmentation procedures, or injected subjacent to the corrugator muscles in procedures designed to weaken these muscles and reduce forehead wrinkles.

As described herein, a tissue processor device may be used to render strips or sheets of autograft dermis, allograft dermis, or EBM into a morsellized, cubed, or particulate form, suitable for injection via a syringe and needle or via direct tamponade insertion. In one embodiment, as illustrated in FIG. 3, the tissue processor operates initially by placing the source material 40 (whether strips or sheets of autograft dermis, allograft dermis or EBM) between identical grooved plates 42 and 44 operatively positioned at right angles at each other. The plates 42, 44, one of which is illustrated in FIG. 3B, may be made from any suitable material. In one embodiment, composite stainless steel plates may be used having a high molecular weight plastic plate 43, 45 on the inner tissue-contact side to minimize blade wear. A third plate 48, (“backing plate”), made of a suitable material such as a composite steel or high molecular weight plastic, includes fingers 49 that are adapted to interdigitate into the grooves 41 of the outer surface of either grooved plate 42, 44, acting to eliminate tissue prolapse during cutting. Only one backing plate is needed, although two can be used. The plate 48 is preferably used, first on one side, and then on the other to support the material being micronized. The protection plates 43, 45 also have grooves 39.

The opposing outer plates 42, 44 immobilize the material and have grooves spaced at various distances depending on the desired size of the particulate material. The plates can range in size from 5×5 cm in width to 100×100 cm in width to accommodate various EBM or tissue sheet sizes. Separations between each parallel groove can range from 0.5 mm to 2 mm depending on the particulate size desired. The plates and corresponding grooves, placed at right angles to one another, act as guides for cutting knives 50, used serially or multiple knives used in parallel. These knives first cut along the grooves of one of the two outer plates (plate 42), rendering multiple small strips of material in between the plates. Thereafter, the backing plate 48, is placed on the opposite outer plate (plate 44), with the fingers 49 then interdigitating with the grooves of that outer plate, yielding a completely flat surface to cut on, acting to prohibit material prolapse through the grooves. Once cuts are made along the grooves of plate 42, the backing plate is switched to interdigitate into the grooves of plate 44, and the same parallel cuts are made on outer plate 44, albeit at right angles to the first plate. In this manner, the particulate material between the plates is rendered fragmented or cubed, and thus suitable for injection or for direct tamponade insertion.

In certain embodiments, the systems and methods disclosed herein may be used to place EBM subdermally or intramuscularly via needle insertion through the skin to provide volume enhancement of the face in developmental maladies of facial fat and muscle volume atrophy. Such morsellized sub dermal fillers can take the form of cubes, spheres, or random shapes of EBM, ranging in volume between 0.001 mm³ and 27 mm³. The morsellized EBM is placed via standard syringe injection after hydration with sterile saline beneath the skin of the face in volume deficient areas. Such conditions include but are not limited to Romberg's Hemifacial Atrophy, and facial lipodystrophy associated with HIV treatment, namely protease inhibitor therapy.

In certain other embodiments, the systems and methods disclosed herein may be used to place EBM subdermally via needle insertion through the skin in the nasal region to provide volume enhancement of the nose in situations where nasal grafting is desired to provide volume enhancement. The morsellized EBM is placed via standard syringe injection after hydration with sterile saline. Such morsellized sub dermal fillers can take the form of cubes, spheres, or random shapes of EBM, ranging in volume between 0.001 mm³ and 27 mm³.

In certain further embodiments, the systems and methods disclosed herein may be used to place injectable morsellized, cubed, or particulate EBM subdermally via needle insertion in the cheek and peri-orbital region to provide volume enhancement of the cheek and peri-orbital soft tissues in situations where cheek and lower eyelid volume enhancement is desired to provide aesthetic enhancement. The morsellized EBM is placed via standard syringe injection after hydration with sterile saline. Such morsellized sub dermal fillers can take the form of cubes, spheres, or random shapes of EBM, ranging in volume between 0.001 mm³ and 27 mm³.

In certain other embodiments, the systems and methods disclosed herein may be used to place injectable morsellized, cubed, or particulate EBM submucosally and/or intramuscularly via needle insertion in the peri-urethral soft tissues to provide bulking and volume enhancement of peri-urethral soft tissues in situations where bulking materials are desired in the treatment of female urinary stress incontinence and in cases of post-prostatectomy urinary incontinence. The morsellized EBM is placed via standard syringe injection after hydration with sterile saline. Such morsellized sub dermal fillers can take the form of cubes, spheres, or random shapes of EBM, ranging in volume between 0.001 mm³ and 27 mm³.

In still other embodiments, the systems and methods disclosed herein may be used to place injectable morsellized, cubed, or particulate EBM submucosally and/or intramuscularly via needle insertion in the peri-laryngeal and pharyngeal soft tissues to provide bulking and volume enhancement of the peri-laryngeal and pharyngeal soft tissues in situations where bulking materials are desired in the treatment of vocal cord paralysis and in speech disorders related to cleft palate deformities. The morsellized EBM is placed via standard syringe injection after hydration with sterile saline. Such morsellized sub dermal fillers can take the form of cubes, spheres, or random shapes of EBM, ranging in volume between 0.001 mm³ and 27 mm³.

In other embodiments, a tamponade technique is used to place morsellized EBM in the form of cubes, ellipses, pre-cut or random shapes and morsellized EBM particles, where the morsellized EBM is a volume filler inserted using a tamponade device, such as is described in more detail below. In this context EBM may be used as a subdermal volume filler in cosmetic and reconstruction. It is understood in the art that this tamponade insertion technique differs from a syringe injection in that the method of insertion in this technique does not depend on flow or the development of pressure within in a syringe. The tamponade insertion device technique involves the extrusion of pre-shaped or particulate EBM out of pre-filled insertion needles. The EBM is therefore extruded out the tip of the needle via a direct plunger effect.

Further disclosed is a device for performing direct tamponade insertion of morsellized, cubed, pre-shaped or particulate EBM, allograft dermis or autograft dermis in the subdermally, submucosally or intramuscularly for volume enhancement. Volume enhancement includes cosmetic subcutaneous, subdermal and intramuscular volume enhancement of the face, lips and cheeks, volume enhancement for the treatment of age related and pathologic soft tissue atrophy, cosmetic volume enhancement of the breast and penis, and bulking of the periurethral soft tissues for the treatment of urinary stress incontinence and for urinary incontinence post prostatectomy, or any other type of soft tissue volume reconstitution, augmentation or reconstruction.

In one embodiment, as illustrated in FIG. 4, a tamponade device is disclosed comprising two components: 1) a thin walled needle 60 made from stainless steel or any other suitable material ranging in size from 8 gauge to 18 gauge and ranging in length from 5 centimeters to 25 centimeters having a handle 62 mounted on the back or proximal end of the needle that allows for digital backwards pressure; and 2) a tamping plate or plunger 70 made from stainless steel or any other suitable material having a plunger at its distal end and an opposing thumb plate 72 at its proximal end to allow for thumb pressure, as illustrated in FIG. 4. In this embodiment, needles may be pre-filled with EBM or manually filled with morsellized, cubed, elliptical, pre-shaped EBM. The tamping plate 70 is operatively connected to the thin-walled needle 60 so that an operator's digital pressure on the thumb plate 72 is transmitted to the material filling the thin-walled needle component so that the material is pushed through the needle component and is extruded out the front (distal) end of the needle component. To effect this, the width of the tamping component is such that it fits exactly within the lumen of the needle component, and the length of the tamping component is such that when fully engaged into the full length of the needle component, the tamp end is flush with the end of the needle component.

In the construction of the tamponade device, audible or tactile feedback may be provided to the operator indicating the volume of filler material that has been inserted. For example, transverse depressions, ridges or similar notches or protrusions may be placed along the length of the tamping component with the depressions, notches or ridges, spaced at intervals ranging from 1 mm to 3 mm. These detent means interdigitate with a cam on the back (proximal) end of the needle component produce audible and mechanical clicking at regular intervals as the tamping component is advanced, thereby to provides the operator with feedback as to the relative volume of material inserted. With reference to FIG. 4 it is noted that the plunger 70 may be provided with notches 74 on its inner surface that selectively engage with a ball 76, shown somewhat schematically in FIG. 4. This notch and ball arrangement forms a camming action that provides the user with some degree of tactile feedback as the device is used.

In use, the tamponade device described herein provides for the direct tamponade insertion of cubed, pre-shaped, elliptical, morsellized or particulate autograft dermis, allograft dermis, or EBM into the subdermal, submucosal or intramuscular space within the body for soft tissue volume enhancement. In use, the distal end of the needle component is inserted into an anatomic space identified for volume supplementation, and digital pressure is applied to the proximal end of the tamping component to extrude filler material comprising pre-shaped, cubed, morsellized or particulate EBM, allograft or autograft via a direct plunger effect.

In certain embodiments of the systems and methods disclosed herein, morsellized, cubed, pre-shaped or particulate EBM may be placed subdermally or intramuscularly via direct surgical insertion through the skin via a tamponade device. Such morsellized subdermal fillers can take the form of cubes, spheres, or randomly cut shapes of EBM, individual pieces ranging in volume between 0.001 mm³ and 27 mm³. The morsellized EBM is placed via tamponade insertion subjacent to the nasolabial folds for treatment of depressions of the nasolabial folds, inserted beneath the lip vermillion via stab incisions in the commissures as currently performed using auto fat grafting in lip augmentation procedures, and inserted subjacent to the corrugator muscle in procedures designed to weaken these muscles and reduce forehead wrinkles.

In certain embodiments of the systems and methods disclosed herein, morsellized, cubed, pre-shaped or particulate EBM may be placed subdermally or intramuscularly via tamponade insertion through the skin to provide volume enhancement of the face in developmental maladies of facial fat and muscle volume atrophy. Such morsellized, sub dermal fillers can take the form of cubes, elliptical shapes, spheres, or random shapes of EBM, individual pieces ranging in volume between 0.001 mm³ and 27 mm³. The morsellized EBM is placed via tamponade insertion beneath the skin of the face in volume deficient areas. Such conditions include but are not limited to Romberg's Hemifacial Atrophy, and facial lipodystrophy associated with HIV treatment, namely protease inhibitor therapy.

In certain embodiments of the systems and methods disclosed herein, morsellized cubed, pre-shaped or particulate EBM may be placed subdermally via direct tamponade insertion through the skin in the nasal region to provide volume enhancement of the nose in situations where nasal grafting is desired to provide volume enhancement. Such morsellized sub dermal fillers can take the form of cubes, elliptical shapes, spheres, or random shapes of EMB, individual pieces ranging in volume between 0.001 mm³ and 27 mm³.

In certain other embodiments of the systems and methods disclosed herein, morsellized cubed, pre-shaped or particulate EBM may be placed subdermally via direct tamponade insertion in the cheek and peri-orbital region to provide volume enhancement of the cheek and peri-orbital soft tissues in situations where cheek and lower eyelid volume enhancement is desired to provide aesthetic enhancement. Such morsellized sub dermal fillers can take the form of cubes, spheres, or random shapes of EBM, individual pieces ranging in volume between 0.001 mm³ and 27 mm³.

In certain embodiments of the systems and methods disclosed herein, morsellized cubed, pre-shaped or particulate EBM may be placed subdermally via direct tamponade insertion in the peri-urethral soft tissues to provide bulking and volume enhancement of peri-urethral soft tissues in situations where bulking materials are desired in the treatment of female urinary stress incontinence and in cases of post-prostatectomy urinary incontinence. Such morsellized sub dermal fillers can take the form of cubes, spheres, or random shapes of EBM, individual pieces ranging in volume between 0.001 mm³ and 27 mm³.

Disclosed herein are systems and devices for preparing EBM into forms suitable for use in the aforesaid methods. Using these systems, devices and related methods, EBM may be morsellized, cubed, or rendered into particulate form, suitable for injection or direct tamponade insertion for volume enhancement. The disclosed device may further be useful for treating autograft dermis or allograft dermis, morsellizing, cubing or rendering these tissues into particulate form for injection or direct tamponade insertion for volume enhancement. The disclosed device may be used to provide reliable cubing, morsellization, and size reduction of EBM, allograft dermis and autologous dermis, such preparations being useful either for injection, for direct tamponade insertion, or for other similar techniques to provide soft tissue volume enhancement. It is understood that standard EBM is derived from the hides of fetal cows and is commercially supplied in sheets as small as 5×6 cm wide and 0.5-1.5 mm thick, and as large as 10×15 cm wide and 0.5-1.5 mm thick. It is also understood that allograft and autograft dermis is harvested in a variety of sizes depending on clinical availability.

In certain other embodiments, a syringe alternatively may be provided pre-filled with various volumes of morsellized, cubed, pre-shaped or particulate EBM, of varying particulate sizes, suitable for hydration and injection subdermally, submucosally or intramuscularly for volume enhancement, for example in the practice of cosmetic and reconstructive surgery, in urology (e.g., for the treatment of incontinence) and in otolaryngology (e.g., for the treatment of pharyngeal disorders, speech disorders, swallowing disorders, or vocal cord abnormalities). FIG. 2 depicts an embodiment of a syringe system for injection of EBM. In an exemplary system, sterile or non-sterile, pre-filled syringes may be provided ranging in volume from 1 cc to 5 cc, containing sterile or non-sterile injectable Embryonic Bovine Collagen Matrix (“EBM”). The injectable EBM can take the form of cubes, elliptical shapes, spheres, or random shapes of EBM, individual pieces ranging in volume between 0.001 mm³ and 27 mm³.

The systems and methods described herein may further be embodied as kits for applying EBM to anatomic spaces for volume supplementation. In certain embodiments, the kits may include a delivery device (e.g., a syringe or a tamponade device), a sterile or non-sterile vial of appropriately sized and shaped EBM, optionally an insertion device such as a tube or a nozzle for filling the delivery device with EBM, and instructions for the use of the kit for tissue volume supplementation. The kit as disclosed herein would be suitable for combination with other pharmacological agents that could be added to the delivery device along with the EBM filler so that the pharmacological agent (e.g., a local anesthetic, an antibiotic or a vasoconstrictor) would be delivered into the tissues simultaneously with the filler.

Clinical Case Examples:

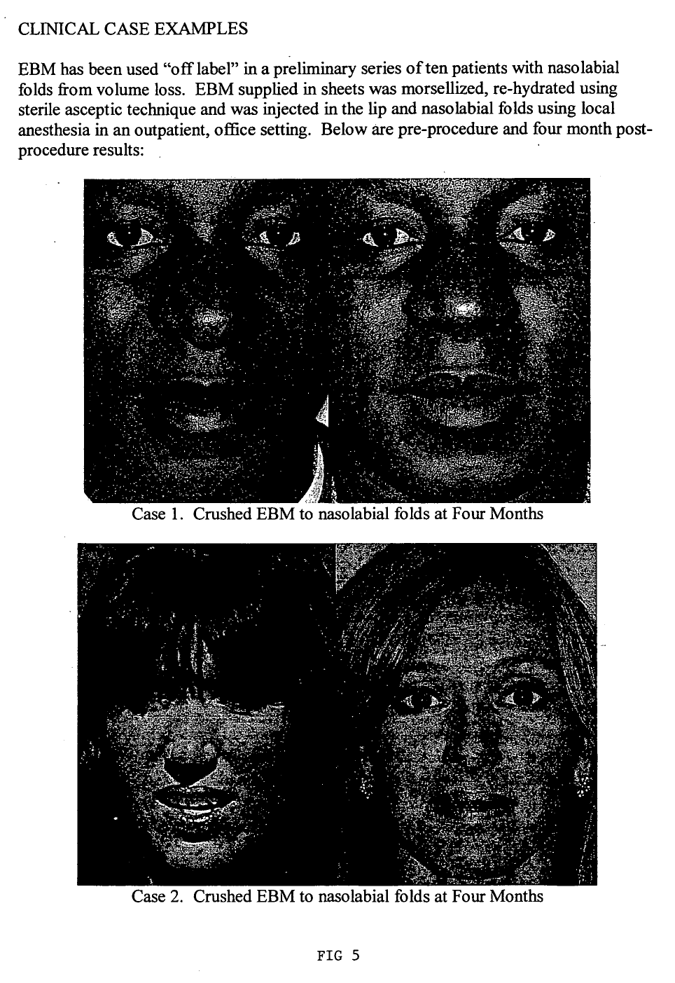

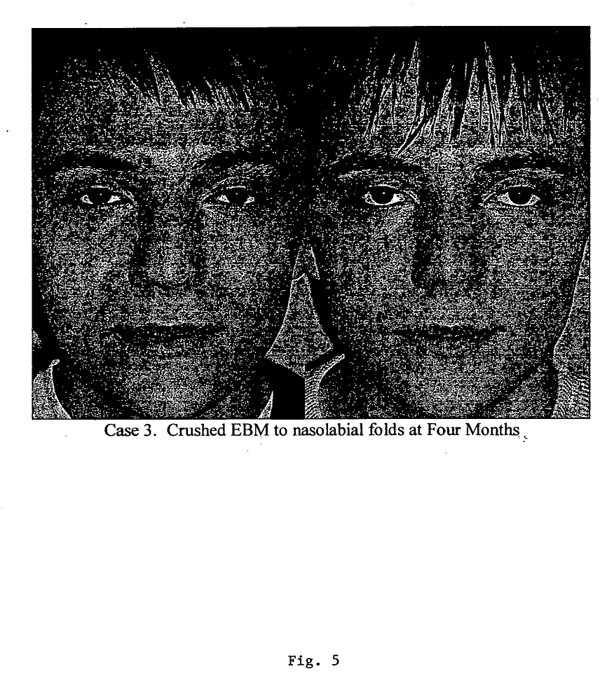

EBM has been used “off label” in a preliminary series of ten patients with nasolabial folds from volume loss. EBM supplied in sheets was morsellized, re-hydrated using a sterile asceptic technique and was injected in the lip and nasolabial folds using local anesthesia in an outpatient, office setting. FIG. 5 are a series of photographs showing the pre-procedure and four month post-procedure results.

Those skilled in the art will recognize, or will be able to ascertain using no more than routine experimentation, numerous equivalents to the systems and methods described herein. Such equivalents are considered to fall within the scope of the present invention and are covered by the following claims. Moreover, the embodiments described herein are intended to exemplify the invention and not to limit it. It is understood that the scope of the present invention is to be construed in accordance with the appended claims.

What is claimed is:

1. A method of treating subcutaneous volume deficiency in the face, lips, cheeks, penis, and peri-urethral areas comprising the steps of: processing sheets of at least one of dehydrated embryonic bovine matrix, autograft dermis or allograft dermis material; morsellizing the material employing manual or machine operated mechanisms, rehydrating the morsellized tissues; and transplanting the re-hydrated tissue into the area being treated via syringe injection.

2. The method of claim 1, further including the steps of morsellizing the dehydrated embryonic bovine matrix; rehydrating the material to enhance flow; forming a passageway through the skin or mucosa; and introducing the morsellized material into the subcutaneous, deep dermal, or periurethral space being treated through the passageway via syringe injection.

3. The method of claim 1, further including the step of adding one or more therapeutic substances to the dehydrated tissue prior to the implantation thereof.

4. The method of claim 3, wherein the therapeutic substances include one or more of the following: growth factors, differentiation factors, hydrogels, polymers, antibiotics, anti-inflammatory medications, or immunosuppressive medications.

5. The method of claim 1, wherein the morsellized material is injected into the area being treated through a needle and syringe or small cannula.

6. The method of claim 1, wherein the morsellized material is percutaneously or transmucosally injected into the area being treated.

7. A method of treating subcutaneous volume deficiency in the face, lips, cheeks, penis, and peri-urethral areas comprising the steps of: processing sheets of at least one of dehydrated embryonic bovine matrix, autograft dermis or allograft dermis material; cubing the material employing manual or machine operated mechanical methods, re-hydrating the cubed tissue; and transplanting the re-hydrated tissue into the area being treated.

8. The method of claim 7, further including the steps of: cubing the dehydrated material; rehydrating the material to enhance flow; forming a passageway through the skin or mucosa; and introducing the cubedmaterial into the subcutaneous, deep dermal, or periurethral space being treated through the passageway via a tamponade introducer.

9. The method of claim 7, further including the step of adding one or more therapeutic substances to the dehydrated tissue prior to the implantation thereof.

10. The method of claim 9, wherein the therapeutic substances include one or more of the following: growth factors, differentiation factors, hydrogels, polymers, antibiotics, anti-inflammatory medications, or immunosuppressive medications.

11. The method of claim 7, wherein the cubed embryonic bovine matrix is transplanted into the area being treated via a tamponade introducer.

12. The method of claim 7, wherein the cubed embryonic bovine matrix is percutaneously or transmucosally transplanted into the area being treated.

13. A method for augmenting volume of a soft tissue, comprising delivering at least one of Embryonic Bovine Collagen Matrix (EBM); autograft dermis or allograft dermis to the soft tissue in the form of at least one of morsellizing, fragmenting, micronizing or cubing.

14. The method of claim 13, wherein the step of delivering to the soft tissue is performed by a tamponade technique.

15. The method of claim 13, wherein the step of delivering to the soft tissue is performed by a syringe injection technique.

16. The method of claim 13, further comprising morsellizing the EBM before delivering it to the soft tissue.

17. The method of claim 13, wherein the soft tissue is selected from the group consisting of subdermal space, subcutaneous space, submucosal space, periurethral space and hypopharyngeal space.

18. A device for morcellizing a soft tissue filler material, comprising two cutting plates attachable at an angle to each other and a cutter operatively connected to the cutting plates, wherein each of the two cutting plates has a series of grooves separated by a predetermined distance, each groove directing the cutter to cut the soft tissue material into fragments of a preselected size.

19. The device of claim 18 including a pair of lining plates disposed between the cutting plates and material.

20. The device of claim 18 including at least one backing plate having fingers for interdigitating into the plate grooves.
